(19) United States
(12) Patent Application Publication
(10) Pub. No.: US 2026/0165882 A1
(43) Pub. Date: Jun. 18, 2026

(54) PROBE-BASED FEMTOSECOND LASER DELIVERY

(71) Applicant: Alcon Inc., Fribourg (CH)

(72) Inventors: Steven T. Charles, Memphis, TN (US); Mikhail Ovchinnikov, Dana Point, CA (US)

(21) Appl. No.: 19/408,617

(22) Filed: Dec. 4, 2025

Related U.S. Application Data
(60) Provisional application No. 63/734,296, filed on Dec. 16, 2024.

(51) Int. Cl. A61F 9/008; H01S 3/00; H01S 3/067; H01S 3/0933

(52) U.S. Cl. CPC A61F 9/008; H01S 3/0071; H01S 3/06758; H01S 3/0933; A61F 2009/00863; A61F 2009/00874

(57) ABSTRACT

The present disclosure generally relates to surgical instruments for ophthalmic surgical procedures, and more specifically, surgical instruments that are capable of providing femtosecond laser pulses. In some aspects, a surgical instrument includes a body and a power amplifier disposed within the body. The power amplifier is configured to receive laser pulses from a laser source external to the body, and to deliver amplified laser pulses to an optical fiber extending toward a distal end of the body. The surgical instrument further includes an optical element disposed near the distal end. The optical element is configured to focus the amplified laser pulses.

### INTRODUCTION

Anatomically, the human eye is divided into two distinct regions: the anterior segment and the posterior segment. The anterior segment includes the lens and extends from the outermost layer of the cornea to the posterior of the lens capsule. The posterior segment of the eye includes the anterior hyaloid membrane and all of the ocular structures behind it, such as the vitreous humor, retina, choroid, and the optic nerve.

Ophthalmic surgical procedures are often classified as anterior segment surgical procedures, posterior segment procedures, or combined anterior segment and posterior segment procedures (i.e., “combined procedures”). Anterior segment surgical procedures typically include surgeries performed on the iris and/or lens, such as cataract surgery. Posterior segment surgical procedures typically include retinal and vitreoretinal surgeries. In certain cases, a patient may have pathologies of the eye requiring both anterior and posterior procedures; in such cases, a combined procedure may be performed.

Conventional surgical instruments for anterior, posterior, and/or combined procedures often use ultrasonic energy or laser radiation to disintegrate tissue prior to aspiration. However, these approaches typically exert negative effects on surrounding tissues in the eye, which may include mechanical, thermal, and non-thermal effects.

### SUMMARY

The present disclosure generally relates to surgical instruments for ophthalmic surgical procedures, and more specifically, surgical instruments that are capable of providing femtosecond laser pulses.

In some aspects, a surgical instrument is provided. The surgical instrument includes a body and a power amplifier disposed within the body. The power amplifier is configured to receive laser pulses from a laser source external to the body, and to deliver amplified laser pulses to an optical fiber extending toward a distal end of the body. The surgical instrument further includes an optical element disposed near the distal end. The optical element is configured to focus the amplified laser pulses.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope, and may admit to other equally effective aspects.

FIG. 1 illustrates a perspective view of an exemplary surgical instrument, according to one or more aspects.

FIG. 2A illustrates a plan view of a portion of the surgical instrument of FIG. 1, according to one or more aspects.

FIG. 2B illustrates a stylized longitudinal cross-sectional view of a portion of a distal tip of the surgical instrument of FIG. 1, according to one or more aspects.

FIG. 3A illustrates an exemplary surgical system having a distributed chirped pulse amplification laser system, according to one or more aspects.

FIG. 3B illustrates an exemplary implementation of the surgical instrument of FIG. 1, according to one or more aspects.

FIG. 4A illustrates a plan view of the surgical instrument having an optical element and a photonic bandgap fiber with a common optical axis, according to one or more aspects.

FIG. 4B illustrates a plan view of the surgical instrument having an actuator coupled with a distal end of photonic bandgap fiber, according to one or more aspects.

FIG. 4C illustrates a plan view of the surgical instrument with an optical element having an offset optical axis, according to one or more aspects.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

The present disclosure generally relates to surgical instruments for ophthalmic surgical procedures, and more specifically, surgical instruments that are capable of providing femtosecond laser pulses.

In some aspects, the surgical instrument includes a body having a photonic bandgap fiber extending through the body toward a distal end thereof. The photonic bandgap fiber, often doped with ions of rare-earth elements (e.g., erbium, ytterbium, thulium, neodymium, praseodymium, and so forth) or transition metals (e.g., titanium, divalent or trivalent chromium, and so forth), acts as an optical gain medium and facilitates amplification to laser pulses that are provided by an external (or remote) laser source. In some aspects, the laser source is a femtosecond laser having a megahertz (MHz) repetition rate. In this way, the surgical instrument itself may be part of a distributed laser system.

In some aspects, the surgical instrument further includes an optical element, such as a microlens or a metalens, that is configured to focus the amplified laser pulses. In some aspects, the surgical instrument further includes an actuator that is coupled with the optical element or a distal end of the photonic bandgap fiber. In this way, the actuator is configured to steer the amplified laser pulses, which allows the laser pulses to be delivered effectively at the higher repetition rate.

In some aspects, a surgical system has a Master Oscillator Power Amplifier (MOPA) configuration, where the power amplifier is disposed in the surgical instrument, and the master oscillator is remote from the surgical instrument (such as being disposed in a surgical console). A photonic bandgap fiber extends from the master oscillator (of the console) to the body of the surgical instrument. The power amplifier is implemented as a diode-pumped, large mode area (LMA) fiber. A stretcher having a dispersive element is disposed in the body between the photonic bandgap fiber and the power amplifier. A compressor having a dispersive element with an opposite dispersion is disposed in a probe of the surgical instrument. In this way, the surgical system provides chirped pulse amplification.

The implementations of the surgical instrument described herein provide a number of benefits over conventional approaches. For example, the surgical instrument may exert reduced effects on other structures of the eye during ophthalmic procedures, as the femtosecond laser pulses produce substantially no thermal effects, and the surgical instrument may deliver laser pulses with greater spatial coherence. During cataract procedures, the surgical instrument may support the delivery of femtosecond laser pulses in a plane that is near-parallel to the posterior capsule, and further may be delivered behind the iris. The surgical instrument may further support the delivery of femtosecond laser pulses to the trabecular meshwork (e.g., spongy tissue located near the cornea through which aqueous humor flows out of the eye). The surgical instrument further provides a direct approach to severing vitreous collagen fibers during vitrectomy procedures.

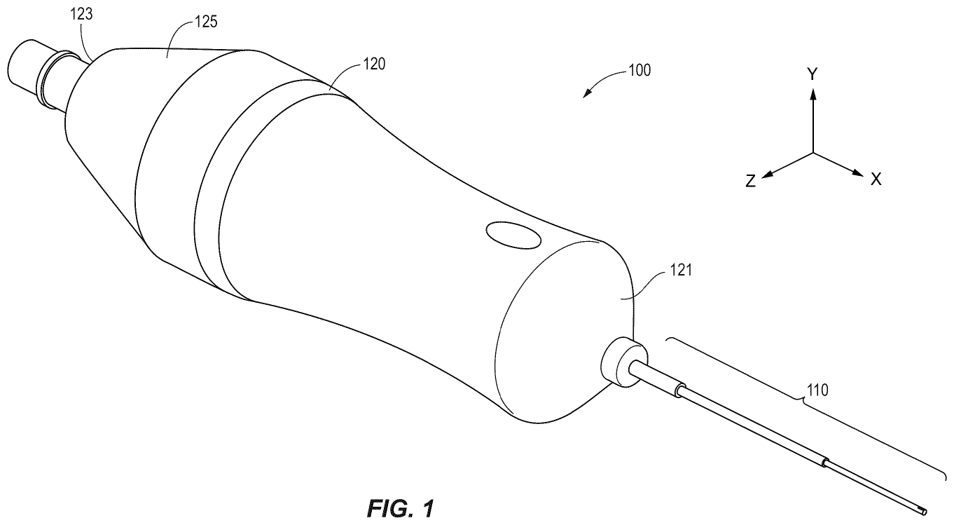

FIG. 1 illustrates a perspective view of an exemplary surgical instrument 100 according to one or more aspects. As depicted in FIG. 1, the surgical instrument 100 includes a probe 110 and a body 120. The probe 110 is partially and longitudinally disposed through a distal end 121 of the body 120 and may be directly or indirectly attached thereto within an interior chamber of the body 120. Note that, as described herein, a distal end or portion of a component refers to the end or the portion that is closer to a patient's body during use thereof. On the other hand, a proximal end or portion of the component refers to the end or the portion that is distanced further away from the patient's body.

In some aspects, the body 120 is implemented as a hand piece having an outer surface configured to be held by a user, such as a surgeon. For example, the body 120 may be ergonomically contoured to substantially fit the hand of the user. In some aspects, the outer surface may be textured or have one or more gripping features formed thereon, such as one or more grooves and/or ridges. The body 120 may be made from any materials commonly used for such instruments and suitable for ophthalmic surgery. For example, the body 120 may be formed of a lightweight aluminum, a polymer, or other suitable material. In some aspects, the body 120 may be sterilized and used in more than one surgical procedure, or it may be a single-use device.

The body 120 further provides one or more ports 123 (e.g., one port 123 is depicted in FIG. 1) at a proximal end 125 thereof for one or more supply lines to be routed into one or more interior chambers defined in the body 120. For example, the port 123 may provide a connection between the body 120 and a vacuum line of a vacuum source for aspiration. In some aspects, the ports 123 may also provide a connection to (or a pass-through for) an optical fiber cable that couples to one or more external laser sources for providing laser pulses. For example, the port 123 may provide a pass-through for a photonic bandgap fiber that is optically coupled with an external laser source and that extends through the body 120 toward the distal end 121.

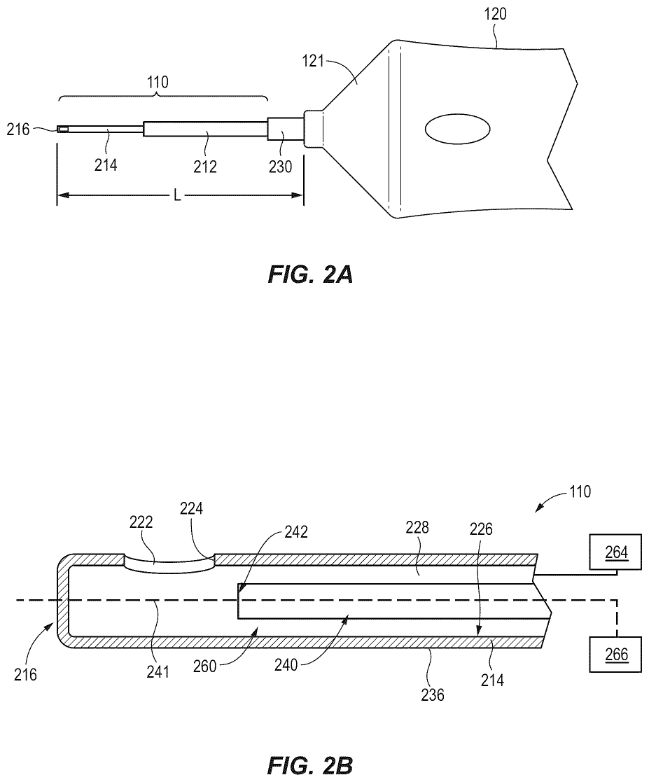

FIG. 2A illustrates a plan view of a portion of the surgical instrument 100, and more specifically, the attachment of the probe 110 with the distal end 121 of the body 120. As shown, the probe 110 may be implemented as an elongated laser cutting member that may be inserted into an eye (e.g., through an insertion cannula) for performing vitreoretinal surgical procedures, such as an aspirating or non-aspirating vitrectomy. Other types of ophthalmic procedures using laser delivery are also contemplated, such as cataract surgery, trabecular surgery for glaucoma, and so forth. The probe 110 may thus be formed of materials suitable for minimally invasive ophthalmic procedures. In some aspects, the probe 110 includes one or more sections that are formed of materials that are optically transmissive of laser light. For example, the probe 110 may include one or more sections formed of a translucent or transparent material, such as a plastic and/or a polymeric material. The probe 110 may further include one or more sections formed of more conventional surgical-grade materials, such as stainless steel and/or aluminum. In other aspects, the probe 110 may define one or more openings (e.g., in an opaque surgical-grade material) allowing the transmission of laser light therethrough.

In some aspects, the probe 110 has a length L between about 15 mm (millimeters) and about 30 mm, but may have a larger or smaller length in other aspects. In some aspects, the probe 110 may comprise a hollow tube having an outer diameter less than about 20 gauge. In some aspects, the probe 110 is segmented into two or more portions (e.g., regions or segments) having outer diameters of differing sizes. For example, as shown in FIG. 2A, the probe 110 may include a proximal portion 212 having a larger outer diameter than a distal portion 214 of the probe 110 that terminates at a distal tip 216. In some aspects, the proximal portion 212 has an outer diameter of about 23 gauge and the distal portion 214 has an outer diameter of about 25 gauge. In some aspects, the proximal portion 212 has an outer diameter of about 25 gauge and the distal portion 214 has an outer diameter of about 27 gauge. In some other aspects, the proximal portion 212 has an outer diameter of about 27 gauge and the distal portion 214 has an outer diameter of about 29 gauge. In some aspects, the proximal portion 212 functions as an infusion portion and is configured to direct infusion fluid into the operating space adjacent the probe during use thereof. The proximal portion 212 may thus include one or more coaxial infusion ports concentrically disposed around the distal portion 214 and fluidly connected to a fluid source through the body 120. Delivery of infusion fluid to the interior eye during vitreoretinal surgery enables the maintenance of intraocular pressure, thereby preventing the eye from collapsing during the surgical procedure.

In some aspects, the surgical instrument 100 further includes a stiffener 230 fixedly or slidably coupled to, and substantially surrounding at least a portion of, the probe 110. For example, the stiffener 230 is slidably coupled to an exterior surface 236 (shown in FIG. 2B) of the probe 110 and may extend from and retract into the body 120. The stiffener 230 may be adjustable relative to the probe 110, enabling a user to position the stiffener 230 at different points along the length L of the probe 110 exterior to the body 120. Accordingly, a user may selectively adjust the level of stiffness of the probe 110 by re-positioning the stiffener 230 relative to the distal tip 216, thereby manipulating the amount of support provided to the probe 110 and stabilizing the surgical instrument 100 during the use thereof.

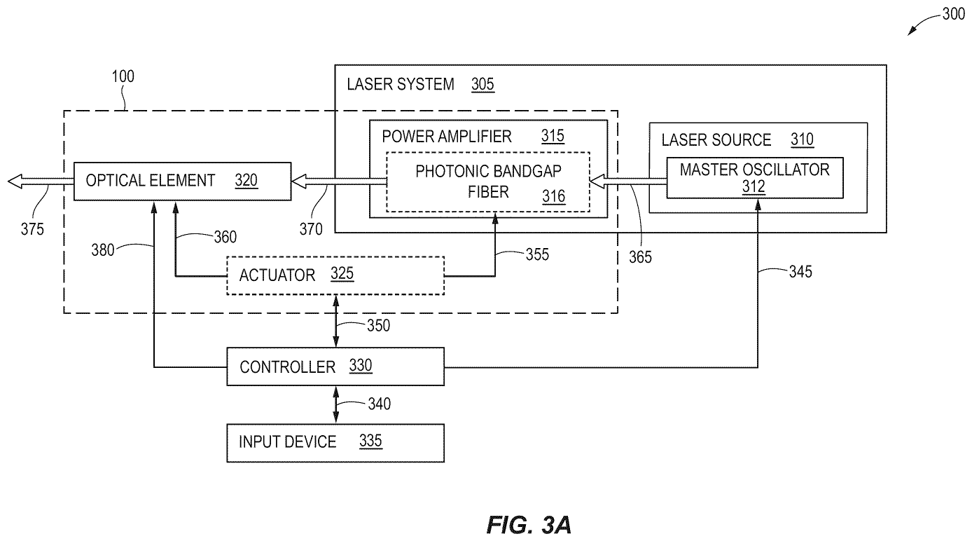

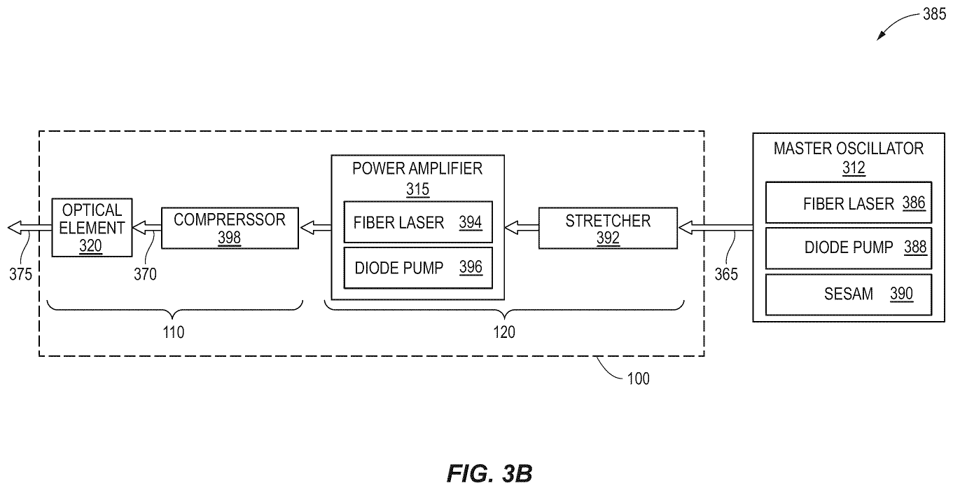

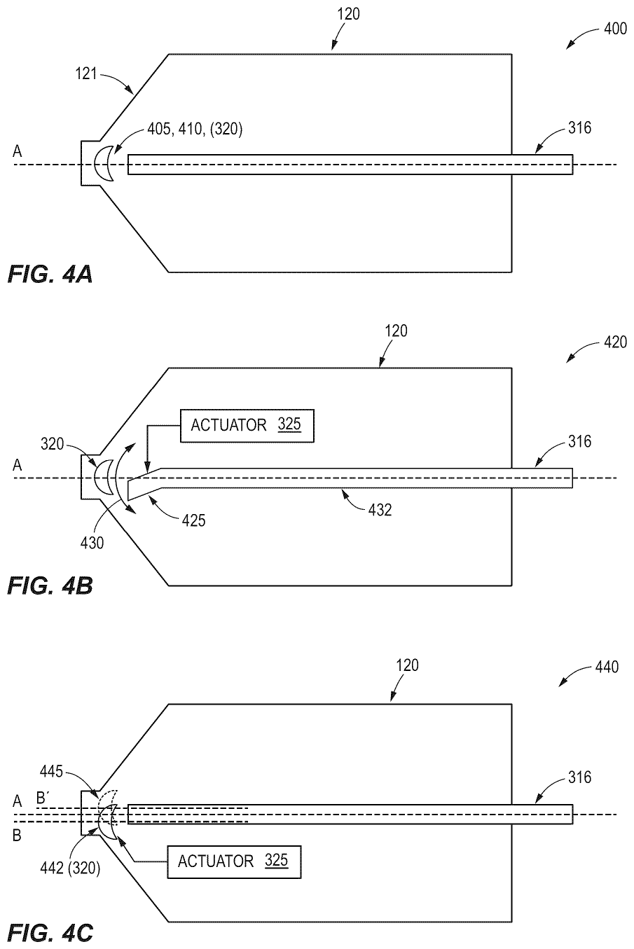

As described above, in some aspects, the surgical instrument 100 provides a photonic bandgap fiber that receives laser pulses from an external laser source, and amplifies the laser pulses for delivery through the distal tip 216 of the probe 110. In some aspects, the surgical instrument 100 further provides an optical element, such as a microlens or a metalens, that focuses the amplified laser pulses for delivery. In some aspects, the external laser source is a femtosecond laser, providing femtosecond-range laser pulses with a megahertz (MHz) repetition rate (e.g., a number of pulses emitted per second). An example implementation of a surgical system having a distributed chirped pulse amplification laser system is described below with respect to FIG. 3. Some example implementations of the surgical instrument 100 are described below with respect to FIGS. 4A, 4B, and 4C.

FIG. 2B illustrates a stylized longitudinal cross-sectional view of a portion of the distal tip 216 of the surgical instrument 100 of FIG. 1, according to one or more aspects. More specifically, the distal portion 214 of the probe 110 is depicted, which includes a main lumen 260 and a port 222 near the distal tip 216. In one example, the main lumen 260 has a substantially circular cross-section, although other cross-sectional geometries are also contemplated. The port 222 is located at the distal tip 216 of the distal portion 214, and in some aspects may be sized and shaped to allow vitreous collagen fibers to enter the main lumen 260 during a vitrectomy procedure. In some examples, a vacuum source 264 is fluidly connected with the port 222 through the main lumen 260, and the vitreous collagen fibers may be aspirated into the main lumen 260 through the port 222. As further described below, a laser source 266 generates laser pulses 241 that are transmitted longitudinally through the main lumen 260 (e.g., through an optical fiber 240) to sever the vitreous collagen fibers that enter the port 222. In an alternate aspect, the laser source 266 may be configured to have a focal spot beyond the distal tip 216, such that the laser pulses 241 may sever the vitreous collagen fibers, which are then aspirated through the port 222 into the main lumen 260.

The optical fiber 240 may be designed to operate as an optical waveguide and propagate the laser pulses 241 through a terminal end 242 thereof. In some aspects, the optical fiber 240 includes a photonic bandgap fiber (one type of photonic crystal fiber), discussed below with respect to FIG. 3. In other aspects, the optical fiber 240 differs from the photonic bandgap fiber. For example, the optical fiber 240 may encompass other types of photonic crystal fibers (such as holey fibers and Bragg fibers), as well as conventional optical fiber implementations. The characteristics of the laser pulses 241 propagated through the optical fiber 240 are such that the laser pulses 241 cause disruption of the vitreous collagen fibers within the path of the laser pulses 241. Disruption refers to the breaking down of the tissue by rapid ionization of molecules thereof. In some aspects, the laser pulses 241 are an ultraviolet (“UV”) (e.g., <350 nm (nanometers)) laser light. In other aspects, the laser pulses 241 are an argon blue-green laser light (e.g., 488 nm), a neodymium-doped yttrium aluminum garnet (“Nd-YAG”) laser light (e.g., 532 nm) such as a frequency-doubled Nd-YAG laser light, a krypton red laser light (e.g., 647 nm), a diode laser light (e.g., 805-810 nm), or any other suitable type of laser light for ophthalmic surgery.

Photonic crystal fibers generally employ a micro-structured arrangement of material in a background material of different refractive indexes. The background material may be undoped silica, and a low-index region may be provided by air voids extending along the length of the photonic crystal fiber. Photonic crystal fibers may be implemented as high-index guiding fibers or low-index guiding fibers. High-index guiding fibers guide light in a solid core region by the Modified Total Internal Reflection (M-TIR) principle (similar to conventional optical fibers), where the total internal reflection results from the lower effective index in the micro-structured air-filled region. Low-index guiding fibers guide light using the photonic bandgap effect, where the light is confined to the low-index core region as the photonic bandgap effect hinders propagation in the micro-structured cladding region.

In some aspects, the laser source 266 produces the laser pulses 241 in the femtosecond range. In some aspects, the laser source 266 produces the laser pulses 241 with a repetition rate (or “pulse rate”) in a megahertz (MHz) range. This range can effectively provide disruption of the vitreous body. Other repetition rate ranges can also provide disruption and are thus contemplated as well. In some aspects, the laser source 266 may produce continuous coherent laser pulses 241. For example, the laser source 266 may produce continuous coherent laser pulses 241 at low power.

In certain aspects, the optical fiber 240 is disposed within the main lumen 260 and terminates at the terminal end 242 near the port 222 such that the laser pulses 241 projecting from the optical fiber 240 will be projected across the port 222 with sufficient power to sever vitreous collagen fibers located within the distal tip 216 near the port 222, and/or beyond the distal tip 216. In the aspect depicted in FIG. 2B, the optical fiber 240 is rigidly suspended within the main lumen 260 such that the optical fiber 240 is separated from an interior sidewall 226 of the probe 110 and the optical fiber 240 is circumferentially surrounded by a space 228. The space 228 formed between the optical fiber 240 and the interior sidewall 226 of the probe 110 provides a coaxial path for aspiration of severed vitreous collagen fibers through the probe 110. In some aspects, the optical fiber 240 may be centrally disposed within the main lumen 260 such that a radial distance between the interior sidewall 226 and the optical fiber 240 is uniform along a circumference of the optical fiber 240.

In some aspects, the laser pulses 241 have a diameter or width that is substantially smaller than a diameter or width of the port 222. For example, the port 222 may have a diameter or width between about 200 μm (micrometers) and about 500 μm, such as between about 250 μm and about 450 μm, such as about 300 μm. The laser pulses 241 may have a diameter or width between about 5 μm and about 50 μm, such as between about 10 μm and about 40 μm, such as between about 15 μm and about 30 μm. In some examples, the laser pulses 241 are scanned across the port 222.

FIG. 3A illustrates an exemplary surgical system 300 having a distributed chirped pulse amplification laser system (“laser system”) 305, according to one or more aspects. The features of the surgical system 300 may be used in conjunction with other aspects. For example, the surgical system 300 may be used with the surgical instrument 100 of FIG. 1.

The surgical system 300 includes the laser system 305, the surgical instrument 100, a controller 330, and an input device 335. The controller 330 is an electronic device in communication with various components of the surgical system 300, as will be discussed in greater detail below. The input device 335 is also an electronic device in communication with the controller 330.

As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network. An electronic device may be implemented with any suitable form factor, whether relatively static in nature (e.g., mainframe, computer terminal, server, kiosk, workstation) or mobile (e.g., laptop computer, tablet, handheld, smart phone, wearable device). The communicative capability between electronic devices may be achieved using any suitable techniques, such as conductive cabling, wireless transmission, optical transmission, and so forth.

The electronic device includes one or more processors and a memory. The one or more processors are any electronic circuitry, including, but not limited to one or a combination of microprocessors, microcontrollers, application-specific integrated circuits (ASIC), application-specific instruction set processors (ASIP), and/or state machines, that is communicatively coupled to the memory and controls the operation of the system. The one or more processors are not limited to a single processing device and may encompass multiple processing devices.

The one or more processors may include other hardware that operates software to control and process information. In some aspects, the one or more processors execute software stored in the memory to perform any of the functions described herein. The one or more processors control the operation and administration of the electronic device by processing information (e.g., information received from input devices and/or communicatively coupled electronic devices).

The memory may store, either permanently or temporarily, data, operational software, or other information for the one or more processors. The memory may include any one or a combination of volatile or non-volatile local or remote devices suitable for storing information. For example, the memory may include random-access memory (RAM), read-only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory, a disk, a CD (compact disc), or a flash drive. In particular aspects, the software may include an application executable by the one or more processors to perform one or more of the functions described herein.

In some aspects, the controller 330 may be implemented as part of a surgical console that is operably coupled, physically or wirelessly, to a number of user interfaces, including one or more foot controllers (one example of the input device 335) and one or more surgical instruments, such as the surgical instrument 100. The surgical console provides one or more port connectors for physically coupling the user interfaces to various components or subcomponents of the surgical console. For example, the surgical instrument 100 may include a photonic bandgap fiber 316 that is connected to a first port connector and optically coupled with a laser source 310 implemented in the surgical console. Further, the surgical instrument 100 may be fluidly coupled with a vacuum source, via a vacuum supply line connected to a second port connector, to enable aspiration of cut vitreous from the patient's eye.

In certain aspects, the surgical console further includes a display for displaying information to the user during operation, e.g., infusion fluid parameters such as infusion fluid flow rates and intraocular pressure, as well as information related to the performance of the surgical instrument 100. In some cases, the display may also incorporate an input device (e.g., a touchscreen overlaid or integrated with display hardware; representing another example of the input device 335) for receiving user input. In some aspects, the input device 335 provides one or more input signals 340 to the controller 330, which may be interpreted by the controller 330 and used to generate control signal(s) that are provided to various components of the surgical system 300.

The laser system 305 includes a laser source 310 and a power amplifier 315. In some aspects, the laser system 305 is implemented as a Master Oscillator Power Amplifier (MOPA) laser system, where the laser source 310 includes a master oscillator 312 that generates a low-power laser (“seed”) signal 365 having one or more signal characteristics (e.g., wavelength, pulse duration, linewidths, and so forth). In some aspects, the low-power laser signal 365 generated by the master oscillator 312 includes femtosecond laser pulses with a MHz repetition rate. In some aspects, the controller 330 specifies the one or more signal characteristics by transmitting a control signal 345 to the laser source 310. The master oscillator 312 may have any suitable implementation. In the example implementation shown in diagram 385 of FIG. 3B, the master oscillator 312 is implemented as a fiber laser 386 pumped by a diode pump 388, and including a semiconductor saturable absorber mirror (SESAM) 390 that provides self-starting passive mode-locking for the fiber laser 386. The fiber laser 386, the diode pump 388, and the SESAM 390 may have any suitable implementation.

The power amplifier 315 receives the low-power laser signal 365, amplifies the low-power laser signal 365 to a desired power level, and outputs an amplified laser signal 370. In some aspects, a stretcher 392 may be arranged between the master oscillator 312 and the power amplifier 315, which stretches the laser pulses of the low-power laser signal 365 to cause different wavelength components of the laser pulses to have a desired difference in path length. The stretching of the laser pulses generally causes the intensity of the laser pulses to be significantly less than the intensity limit of the gain medium (e.g., the power amplifier 315). The stretcher 392 may have any suitable implementation, such as a quadratic phase modulator and a dispersive element having a first dispersion (e.g., positive or negative dispersion). Some examples of the dispersive element include a fiber Bragg grating, an anomalous dispersion fiber, and a prism.

The power amplifier 315 may have any suitable implementation. Generally, the power amplifier 315 includes a separate pump source for “pumping” energy into the gain medium to excite rare-earth ions to higher energy states, which then emit additional photons from incoming signal photons (e.g., from the seed signal 365). In the example implementation shown in diagram 385, the power amplifier 315 is implemented as a fiber laser 394 pumped by a diode pump 396. For example, the power amplifier 315 may comprise a cladding-pumped large mode area (LMA) fiber. In some aspects, the power amplifier 315 may be implemented using a photonic bandgap fiber 316, and may optionally comprise hardware (e.g., electronic and/or photonic circuitry) defining one or more amplification stages. As mentioned above, alternate aspects of the fiber laser 394 may encompass other types of optical fibers: photonic crystal fibers (such as holey fibers and Bragg fibers), as well as conventional optical fibers. In some aspects, the power amplifier 315 and the stretcher 392 are disposed in the body 120 of the surgical instrument 100.

In some aspects, a compressor 398 compresses the amplified laser pulses to cause the different wavelength components to return to a same path length. The compressor 398 may have any suitable implementation, such as a dispersive element (e.g., a fiber Bragg grating, an anomalous dispersion fiber, a prism) having a second dispersion opposite to the first dispersion. In this way, the laser system 305 is capable of providing chirped pulse amplification. In some aspects, the compressor 398 and the optical element 320 are disposed in the probe 110 of the surgical instrument 100.

The photonic bandgap fiber 316 may have any suitable implementation, which may encompass solid-core and hollow-core fiber implementations. In some solid-core based aspects, the photonic bandgap fiber 316 includes a core region circumferentially surrounded by a cladding region. The cladding region is formed of material(s) having a first refractive index, e.g., an array of high-index regions that are embedded in silica. The low-power laser signal 365 is confined to the core region by a photonic bandgap established by the geometry of the cladding region. In some aspects, the core region includes a material having a second refractive index less than the first refractive index. The core region may further be doped, e.g., using ytterbium (Yb), erbium (Er), or thulium (Tm) ions, to improve gain characteristics of the photonic bandgap fiber 316.

In other aspects, the photonic bandgap fiber 316 includes a hollow core fiber in which the core region includes an air core and an anti-resonant structure acting as the cladding of the fiber.

Corresponding to the size of the core region of the photonic bandgap fiber 316, the laser pulses of the amplified laser signal 370 exiting the photonic bandgap fiber 316 may have a relatively large diameter or width, such as between about 10 μm and about 20 μm. In some aspects, the surgical instrument 100 further includes an optical element 320 that focuses the laser pulses of the amplified laser signal 370 to have a smaller diameter or width that is suitable for cutting operations. In some aspects, the optical element 320 focuses the laser pulses of the amplified laser signal 370 to output a focused laser signal 375 with a spot size of about 5 μm.

The optical element 320 may have any suitable implementation. In some aspects, the optical element 320 includes a microlens having a diameter less than 1 mm, such as between about 100 μm and about 10 μm. In one example, the microlens may be implemented as a single lens element having a planar surface opposite a (spherical) convex surface providing refraction. In another example, the microlens may be implemented as a ball lens element. In another example, the microlens may be implemented using multiple elements, such as gradient-index lenses or microlens arrays. In some aspects, the optical element 320 includes a metalens comprising numerous meta-atoms (e.g., typically millions of sub-wavelength unit cells) that provide localized phase modulation across an entire metasurface defined by the metalens.

To take advantage of the high repetition rate of the laser pulses during a surgical procedure, the output of the optical element 320 (e.g., the focused laser signal 375) may be actuated or steered. For example, a first laser pulse may break down tissue and form a void (or bubble) at the location. By actuating the output of the optical element 320, subsequent laser pulses may be steered away from the void and directed toward adjacent tissue, improving the effectiveness of the surgical instrument 100.

In some aspects, the surgical instrument 100 further includes an actuator 325 having any suitable implementation. Some examples of the actuator 325 include a motor (e.g., a stepper motor), a piezoelectric actuator, a microelectromechanical system (MEMS) actuator, and so forth. The actuator 325 may be operably coupled to the optical element 320 through a physical connection 360, and/or to a distal end of the photonic bandgap fiber 316 through a physical connection 355. Based on control signal(s) 350 provided by the controller 330, the actuator 325 may supply linear motion, circular motion, elliptical motion, etc. to the coupled component(s). In some alternate aspects, the controller 330 may provide control signal(s) 380 to the optical element 320 to control one or more attributes of the optical element 320 to thereby steer the laser pulses.

FIG. 4A illustrates a plan view 400 of a surgical instrument having an optical element 320 and a photonic bandgap fiber 316 with a common optical axis, according to one or more aspects. The features illustrated in the plan view 400 may be used in conjunction with other aspects, e.g., representing one example implementation of the surgical instrument 100 of FIG. 1.

In the plan view 400, the photonic bandgap fiber 316 extends from beyond a proximal end 125 of the body 120, and through the body 120 (e.g., through an interior volume of the body 120) toward the distal end 121 thereof. The photonic bandgap fiber 316 extends longitudinally along an optical axis A. Although not shown, the body 120 may include structures that arrange and/or retain the photonic bandgap fiber 316 with fixed positioning. Further, although the optical axis A is shown as overlapping a midline of the body 120, other implementations (e.g., offset from the midline) are also contemplated.

In some aspects, the optical element 320 includes a microlens 405 having the common optical axis A with the photonic bandgap fiber 316. In other aspects, the optical element 320 includes a metalens 410 having the common optical axis A with the photonic bandgap fiber 316. Although not shown, the body 120 may include structures that arrange and/or retain the optical element 320 with fixed positioning relative to the photonic bandgap fiber 316. In some aspects, the controller 330 may provide control signal(s) 380 to the optical element 320 (e.g., the metalens 410) to control one or more attributes thereof, and may thus steer the laser pulses transmitted through the optical element 320. Further, in some alternate aspects, one or both of the optical element 320 and the photonic bandgap fiber 316 may be disposed in the probe 110 of the surgical instrument 100.

FIG. 4B illustrates a plan view 420 of a surgical instrument having an actuator 325 coupled with a distal end 425 of the photonic bandgap fiber 316, according to one or more aspects. The features illustrated in the plan view 420 may be used in conjunction with other aspects, e.g., representing one example implementation of the surgical instrument 100 of FIG. 1.

In the plan view 420, the photonic bandgap fiber 316 extends from beyond a proximal end 125 of the body 120, and through the body 120 (e.g., through an interior volume of the body 120) toward the distal end 121 thereof. The photonic bandgap fiber 316 extends longitudinally along an optical axis A. Although not shown, the body 120 may include structures that arrange and/or retain a portion 432 of the photonic bandgap fiber 316 (e.g., away from the distal end 425) in position. In some aspects, the optical element 320 has the common optical axis A with the photonic bandgap fiber 316. Although not shown, the body 120 may include structures that arrange and/or retain the optical element 320 in position relative to the portion 432 of the photonic bandgap fiber 316.

In some aspects, the controller 330 may provide control signal(s) 350 to the actuator 325 to position the distal end 425 of the photonic bandgap fiber 316. For example, the actuator 325 may apply force to push or pull the distal end 425 and cause the photonic bandgap fiber 316 to deflect or pivot relative to the optical axis A. For example, the actuator 325 may deflect the distal end 425 of the photonic bandgap fiber 316 along a substantially circular path 430. Other motion of the distal end 425 is also contemplated, such as linear motion. In this way, the alignment of the core of the photonic bandgap fiber 316 is adjusted by the actuator 325 relative to the optical axis A, which steers the laser pulses that are transmitted through the optical element 320.

FIG. 4C illustrates a plan view 440 of the surgical instrument with an optical element 320 having an offset optical axis B, according to one or more aspects. The features illustrated in the plan view 440 may be used in conjunction with other aspects, e.g., representing one example implementation of the surgical instrument 100 of FIG. 1.

In the plan view 440, the photonic bandgap fiber 316 extends from beyond a proximal end 125 of the body 120, and through the body 120 (e.g., through an interior volume of the body 120) toward the distal end 121 thereof. The photonic bandgap fiber 316 extends longitudinally along an optical axis A. Although not shown, the body 120 may include structures that arrange and/or retain the photonic bandgap fiber 316 with fixed positioning.

The optical axis B of the optical element 320 is offset from the optical axis A of the photonic bandgap fiber 316. In some aspects, the controller 330 may provide control signal(s) 350 to the actuator 325 to revolve the optical element 320 about the optical axis A, which adjusts the alignment of the optical axis B relative to the optical axis A and steers the laser pulses that are transmitted through the optical element 320. For example, the positioning of the optical element 320 may range from its current position 442 corresponding to the optical axis B to a furthest position 445 corresponding to the optical axis B′. In alternate aspects, the actuator 325 may apply force to push or pull the optical element 320 linearly within the range of positions.

In summary, aspects of the present disclosure are directed to surgical instruments that are capable of providing femtosecond laser pulses for ophthalmic surgical procedures. In some aspects, the surgical instrument includes a body and a photonic bandgap fiber extending through the body toward a distal end thereof. The photonic bandgap fiber provides amplification to laser pulses that are provided by an external (or remote) laser source, which in some aspects, is a femtosecond laser having a megahertz (MHz) repetition rate. The surgical instrument may further comprise an optical element that focuses the amplified laser pulses, which in some cases steers the amplified laser pulses for improved delivery. The surgical instrument may exert reduced effects on other structures of the eye during ophthalmic procedures, as the femtosecond laser pulses produce substantially no thermal effects, and the surgical instrument may deliver laser pulses with greater spatial coherence.

Although vitreous surgery is discussed as an example of a surgical procedure that may benefit from the described aspects, the advantages of the surgical devices and systems described herein may benefit other surgical procedures as well.

While the foregoing is directed to aspects of the present disclosure, other and further aspects of the disclosure may be devised without departing from the basic scope thereof, and the scope thereof is determined by the claims that follow.

### EXAMPLE ASPECTS

Aspect 1. A system comprising: a surgical instrument; and a chirped pulse amplification laser system comprising: a master oscillator disposed outside the surgical instrument; and a power amplifier comprising a photonic bandgap fiber that is optically coupled with the master oscillator and that extends into the surgical instrument toward a distal end thereof.

Aspect 2. The system of Aspect 1, wherein the surgical instrument comprises: a body, wherein the photonic bandgap fiber is configured to receive laser pulses from the master oscillator, and to amplify the laser pulses; and an optical element disposed near a distal end of the body, the optical element configured to focus the amplified laser pulses exiting the distal end of the body.

Aspect 3. The system of Aspect 2, the surgical instrument further comprising: a probe extending from the distal end of the body to a distal tip of the probe, wherein the distal tip is optically transmissive of the amplified laser pulses.

Aspect 4. The system of Aspect 3, wherein the body defines a first port configured to couple with a vacuum source, and wherein the probe defines a second port near the distal tip that is fluidly connected with the first port.

Aspect 5. The system of Aspect 1, wherein the master oscillator is configured to provide femtosecond laser pulses.

Aspect 6. The system of Aspect 1, wherein the photonic bandgap fiber comprises a hollow core fiber.

What is claimed is:

1. A surgical instrument comprising:
a body;
a power amplifier disposed within the body, the power amplifier configured to receive laser pulses from a laser source external to the body, and to deliver amplified laser pulses to an optical fiber extending toward a distal end of the body; and
an optical element disposed near the distal end and optically coupled with the optical fiber, the optical element configured to focus the amplified laser pulses.

2. The surgical instrument of claim 1,
wherein the power amplifier comprises a fiber laser and a diode pump, and
wherein a photonic bandgap fiber is the fiber laser and the optical fiber.

3. The surgical instrument of claim 2, wherein the optical element comprises a microlens having a common optical axis with the photonic bandgap fiber.

4. The surgical instrument of claim 2, wherein the optical element comprises a metalens having a common optical axis with the photonic bandgap fiber.

5. The surgical instrument of claim 2, further comprising:
an actuator coupled with the optical element or a distal end of the photonic bandgap fiber, the actuator configured to steer the amplified laser pulses according to one or more control signals.

6. The surgical instrument of claim 5, wherein the optical element has a first optical axis offset from a second optical axis of the photonic bandgap fiber, the actuator configured to revolve the optical element about the second optical axis.

7. The surgical instrument of claim 1, wherein the laser source is a femtosecond laser.

8. The surgical instrument of claim 2, wherein the photonic bandgap fiber comprises a hollow core fiber.

9. The surgical instrument of claim 1, further comprising:
a probe extending from the distal end of the body to a distal tip of the probe, wherein the distal tip is optically transmissive of the amplified laser pulses.

10. The surgical instrument of claim 9, wherein the body defines a first port configured to couple with a vacuum source, and wherein the probe defines a second port near the distal tip that is fluidly connected with the first port.

11. A system comprising:
a surgical instrument comprising an optical element disposed near a distal end of the surgical instrument; and
a chirped pulse amplification laser system comprising:
a master oscillator disposed outside the surgical instrument; and
a power amplifier disposed within the surgical instrument, wherein the power amplifier is optically coupled with the master oscillator and with the optical element.

12. The system of claim 11, wherein the power amplifier comprises a photonic bandgap fiber.

13. The system of claim 12, wherein the optical element comprises a microlens or a metalens having a common optical axis with the photonic bandgap fiber.

14. The system of claim 11, wherein the power amplifier is configured to receive laser pulses from the master oscillator, and to provide amplified laser pulses, the surgical instrument further comprising:
an actuator coupled with the optical element or a distal end of an optical fiber disposed within the surgical instrument, the actuator configured to steer the amplified laser pulses according to one or more control signals.

15. The system of claim 14, wherein the optical element has a first optical axis offset from a second optical axis of the optical fiber, the actuator configured to revolve the optical element about the second optical axis.
